(19) United States
(12) Patent Application Publication
(10) Pub. No.: US 2002/0010144 A1
(43) Pub. Date: Jan. 24, 2002

(54) Enhancing the sensitivity of tumor cells to therapies

(76) Inventors: Robert Sobol, Rancho Santa Fe, CA (US); Ruth Gjerset, San Diego, CA (US)

Correspondence Address: LYON & LYON LLP, 633 WEST FIFTH STREET, SUITE 4700, LOS ANGELES, CA, 90071, US

(21) Appl. No.: 09/758,956

(22) Filed: Jan. 10, 2001

Related U.S. Application Data
(63) Continuation of application No. 08/335,461, filed on Nov. 7, 1994.
Application No. 08/335,461 is a continuation-in-part of application No. 08/236,221, filed on Apr. 29, 1994, now abandoned.

(51) Int. Cl. A61K 48/00

(52) U.S. Cl. 514/44

(57) ABSTRACT

A method for enhancing the effect of a cancer therapy by introducing wild-type therapy-sensitizing gene activity into tumor cells having mutant therapy-sensitizing gene activity and subjecting the tumor cells to a cancer therapy such as chemotherapy, radiotherapy, biological therapy including immunotherapy, cryotherapy and hyperthermia.

### CROSS REFERENCE TO RELATED APPLICATION

This application is a continuation of U.S. patent application Ser. No. 08/335,461, titled “ENHANCING THE SENSITIVITY OF TUMOR CELLS TO THERAPIES,” filed Nov. 7, 1994, which was a continuation-in-part of U.S. application Ser. No. 08/236,221, filed May 24, 1994, which is a continuation-in-part application of U.S. application Ser. No. 08/236,221, filed Apr. 29, 1994; the disclosures of the above applications are incorporated herein by reference.

### FIELD OF THE INVENTION

This invention relates to cancer therapies. In particular, this invention relates to a method of enhancing the effect of cancer therapies.

### BACKGROUND OF THE INVENTION

The mainstays of cancer therapy have been surgery, radiation, chemotherapy and biological therapy (see generally, Comprehensive Textbook of Oncology, ed. A. R. Moossor, et al. (Williams & Wilkins, 1991); Cancer: principles and practice of oncology, ed. Vincent T. DeVita, Jr., Samuel Hellman, Steven A. Rosenberg 4th ed. (Philadelphia: J.B. Lippincott Company, 1993)). Radiation therapy, which is also called radiotherapy, uses high energy x-rays, electron beams, radioactive isotopes and other forms of radiation known to those skilled in the art to kill cancer cells without exceeding tolerable doses to normal tissue.

Chemotherapy refers to the use of drugs to kill cancer cells. There are several classes of chemotherapeutic agents with different modes of action. For example, many anti-metabolites share structural similarities with normal cellular components and they exert their effects by inhibiting normal cellular processes. Many alkylating agents are effective against proliferating and non-proliferating cancer cell populations. In general, these drugs bind with the cell's DNA in various ways to prevent accurate replication and/or transcription. Many anti-tumor antibiotics insert themselves into DNA where they induce breaks in the DNA or inhibit transcription. In general, alkaloids inhibit the function of chromosome spindles necessary for cell duplication. Hormone agents such as tamoxifen and flutamide inhibit the growth of some cancers, although their mechanism of action is not completely understood.

In general, biological therapy utilizes agents which are derived from or which beneficially modulate host biological processes. Interferon-alpha and interleukin-2 are two examples of biological therapy agents currently utilized in cancer therapeutics.

Some cancer therapies use modifying agents to enhance the effect of standard treatment methods (see generally, Coleman C N, Glover D J, Turrisi A T. “Radiation and chemotherapy sensitizers and protectors.” Chemotherapy: Principles and practice. Philadelphia: W B Saunders, 225-252, 1989). Chemical modifiers are usually not cytotoxic by themselves but modify or enhance the response of tumor tissue to a standard therapy, e.g., radiation therapy. The effectiveness of a sensitizer is generally expressed as the sensitizer enhancement ratio (SER). The SER is the dose of therapy required to produce a defined level of killing without the sensitizer divided by the dose of therapy required for the same level of cell killing with the sensitizer.

Two examples of clinical approaches to radiation and chemotherapy modification are hypoxic-cell sensitization and thiol depletion. The damage produced by radiation and alkylating agents is in part related to free radical formation in DNA and other critical cellular macromolecules. Thiol compounds prevent DNA free radicals or repair them. If the DNA free radical is exposed to oxygen or an oxygen-mimetic hypoxic cell sensitizer, such as a nitroimidazole, the damage to DNA is fixed, i.e., made irreversible by oxidation. Depletion of thiols by drugs such as buthonine sulfoximine (BSO) also increases the toxicity from radiation and radiomimetic chemotherapeutic agents such as alkylating agents.

### SUMMARY OF THE INVENTION

This invention features a method for treating cancers which are characterized by loss of wild-type therapy-sensitizing gene activity. The method includes introducing into tumor cells a source of wild-type therapy-sensitizing gene activity and subjecting the cells to a cancer therapy. The cancer therapies whose effect may be enhanced by this invention include, but are not limited to, radiotherapy, chemotherapy, biological therapy including immunotherapy, cryotherapy and hyperthermia. The cancers that can be treated by this invention include, but are not limited to, carcinoma, sarcoma, central nervous system tumor, melanoma tumor, leukemia, lymphoma, hematopoietic cancer, ovarian carcinoma, osteogenic sarcoma, lung carcinoma, colorectal carcinoma, hepatocellular carcinoma, glioblastoma, prostate cancer, breast cancer, bladder cancer, kidney cancer, pancreatic cancer, gastric cancer, esophageal cancer, anal cancer, biliary cancer, urogenital cancer, and head and neck cancer.

Thus, this invention features a method of enhancing the effect of a cancer therapy by delivering a source of wild-type therapy-sensitizing gene activity into a tumor cell characterized by loss of wild-type therapy-sensitizing gene activity and subjecting the tumor cell to the cancer therapy.

By “delivering” is meant the use of methods known to those skilled in the art for administering drugs to a mammal. These methods include, but are not limited to, delivering a gene or cDNA of the gene to a tumor cell in a vector delivering a gene or cDNA of the gene to a tumor cell by coupling with a virus capsid, delivering a gene or cDNA of the gene to a tumor cell by coupling with a ligand or by encapsulation in a liposome, correcting a tumor cell gene point mutation or insertion mutation or deletion mutation by recombination techniques, or delivering protein to cells either directly or in hybrid molecules or by encapsulation methods. Other materials and methods that result in the presence of wild-type therapy-sensitizing gene activity within a tumor cell, such as those described in J. Sambrook, E. F. Fritsch, and T. Maniatis, Molecular Cloning: A Laboratory Manual, 2 Ed., Cold Spring Harbor Laboratory Press, Cold Spring Harbor, New York, 1989, and Ausubel et al., Current Protocols in Molecular Biology, 1994, incorporated by reference herein, may also be utilized.

By “therapy-sensitizing gene” is meant a gene or gene product whose loss of normal function or regulation renders cancer cells more resistant to therapy. Restoration of therapy-sensitizing gene function results in increased sensitivity of cancer cells to therapy. In particular, it is meant a gene which may promote apoptosis or whose altered function or regulation contributes to tumorigenesis and therapy resistance, including, but not limited to, tumor suppressor genes such as p53; cell cycle regulatory genes such as cyclins, cyclin dependent kinases (Steel, M., Lancet 343:931-932, 1994), mitogen activated protein kinases (Blenis, J., Proc. Natl. Acad. Sci. 90:5889-5892, 1993: Marshall, C. J., Nature, 367:686, 1994), inhibitors of cell cycle genes such as p16 (multiple tumor suppressor 1) (Kamb et al., Science 264:436-490, 1994); and apoptosis genes such as fas.

A prospective therapy-sensitizing gene may be identified by the method disclosed in the detailed description of the invention for the therapy-sensitizing gene p53, by substituting p53 with the prospective candidate gene. For example, tumor cells are first characterized by routine sequence analysis or other diagnostic assays known to those skilled in the art to contain a mutated gene or mutated messenger RNA encoding the candidate therapy-sensitizing gene to be tested. The normal wild-type coding sequence for such a gene is then subcloned by standard methods known to those skilled in the art into a suitable eukaryotic expression vector containing a selectable marker gene such as the neomycin resistance gene. For example, the normal coding sequence can be amplified by polymerase chain reaction (PCR) from the cDNA of the messenger RNA population of normal fibroblasts, using appropriate primers to the 31 and 51 ends of the coding sequence. Following subcloning into an appropriate eukaryotic expression vector, the vector containing the normal candidate therapy-sensitizing gene of interest can be transfected into the tumor cells expressing the mutated form of the gene. Transfection can be performed by a number of methods known to those skilled in the art, including but not limited to calcium phosphate transfection, lipofection (which uses cationic liposomes), electroporation, and DEAE-dextran facilitated transfection. The transfected cells are expanded in the presence of the appropriate selection agent, such as neomycin. Once the clones have been expanded and selected, they are: 1) characterized to document expression of the candidate therapy-sensitizing gene by routine methods known to those skilled in the art and 2) tested for sensitivity to chemotherapeutic drugs and/or radiation therapy in standard growth assays or clonogenic assays as described in the detailed description of the invention for p53. Increased sensitivity to therapy in multiple clones expressing the candidate therapy-sensitizing gene compared to parental tumor cells indicates that the transfected gene is a therapy-sensitizing gene.

By “wild-type therapy-sensitizing gene activity” is meant the activity of a therapy-sensitizing gene in a normal, non-neoplastic cell. Specifically, it means the ability of the protein or a portion of the protein encoded by the therapy-sensitizing gene to sensitize a tumor cell to a cancer therapy. A therapy-sensitizing protein having one or more “mutations” that does not affect the therapy-sensitizing ability thereof is still considered “wild-type” for the purpose of this invention. The activity is embodied in the protein expressed from the wild-type therapy-sensitizing gene coding sequence or portions thereof.

By “loss of wild-type therapy-sensitizing gene activity” is meant the absence or alteration of normal therapy-sensitizing gene activity such as the presence of a mutant therapy-sensitizing protein, the absence of a wild-type therapy-sensitizing protein or an inhibited wild-type therapy-sensitizing protein in a cell. The difference from normal in therapy-sensitizing activity may be caused by a genetic difference at one or more genetic loci. The genetic differences may be of several different types, including, but not limited to, a point mutation where a single base pair is changed to another base pair, an insertion of one or more base pairs, a deletion of one or more base pairs up to the full length of the therapy-sensitizing gene, fusion of one gene to another, introduction of additional copies of an existing therapy-sensitizing gene, introduction of one or more copies of a non-therapy-sensitizing gene not formerly present, other alterations of gene transcription, translation and protein function known to those skilled in the art, or any combination of the above.

By “tumor cell” is meant a cell arising in an animal in vivo which is capable of undesired proliferation or abnormal persistence or abnormal invasion of tissues.

In a preferred embodiment, this invention introduces a therapy-sensitizing portion of a wild-type therapy-sensitizing protein into a tumor cell, and subjects said tumor cell to a cancer therapy.

By “therapy-sensitizing portion of a wild-type therapy-sensitizing protein” is meant the portion of a wild-type therapy-sensitizing protein that has the ability to sensitize a tumor cell expressing mutant therapy-sensitizing activity to a cancer therapy. The therapy-sensitizing portion of a wild-type therapy-sensitizing protein may be delineated by routine sequence analysis known to those skilled in the art, including, but not limited to, deletion mutations, point mutations and such as described in Unger et al., “Functional domains of wild-type and mutant therapy-sensitizing proteins involved in transcriptional regulation, transdominant inhibition, and transformation suppression,” Molec. Cell. Biol. 13:5186-94, 1994 and J. Sambrook, E. F. Fritsch, and T. Maniatis, Molecular Cloning: A Laboratory Manual, 2 Ed., Cold Spring Harbor Laboratory Press, Cold Spring Harbor, New York, 1989, and Ausubel et al., Current Protocols in Molecular Biology, 1994, incorporated by reference herein.

In another preferred embodiment, this invention introduces a wild type therapy-sensitizing gene, its cDNA, or a portion thereof encoding the therapy-sensitizing gene activity into a tumor cell, expresses the therapy-sensitizing gene, and subjects the tumor cell to a cancer therapy.

In a further preferred embodiment, the therapy-sensitizing gene, its cDNA, or a portion thereof is introduced into the tumor cell by a viral vector selected from the group, including, but not limited to, adenovirus vector, retroviral vector, adeno-associated virus vector, herpes virus vector, vaccinia virus vector and papilloma virus vector. The therapy-sensitizing gene, its cDNA, or a portion thereof can also be introduced into the tumor cell by coupling to a virus capsid or particle through polylysine bridge, conjugating to a ligand such as an asialoglycoprotein or encapsulation in a liposome. The means of introduction into an animal include, but are not limited to, direct injection or aerosolized preparation, intra-arterial infusion, intracavitary infusion and intravenous infusion.

In some instances, the mutated or abnormal therapy-sensitizing activity may reflect abnormally increased gene expression or gene product activity which may be down regulated by transdominant-negative mutants or other down regulation methods known to those skilled in the art.

Other features and advantages of the invention will be apparent from the following detailed description of the invention, and from the claims.

### BRIEF DESCRIPTION OF FIGURES

FIG. 1 shows cisplatin sensitivity of T98G glioblastoma cells (closed circles) and the same cells with wild-type p53 expressed therein, T98Gp53 cells (open circles).

FIG. 2 shows radiation sensitivity of T98G glioblastoma cells (upper curve) and T98Gp53 cells (lower curve).

### DETAILED DESCRIPTION OF THE INVENTION

This invention features a new method of enhancing the effect of cancer therapy by introducing into a tumor cell a source of therapy-sensitizing activity (through the introduction of a gene, a cDNA or a protein), which has been lost from the tumor cell. Examples of such activities include but are not limited to the fas gene, the retinoblastoma gene, the p53 tumor suppressor gene and other tumor suppressor genes, cell cycle regulatory genes and apoptosis genes.

The Tumor Sensitizing Gene P53/s Relevance to Human Cancer

Loss of normal p53 function, either through mutation, deletion or inactivation, is one of the most frequently encountered alterations in human cancer, occurring in some 50% of human cancers (Nigro et al., “Mutations in the p53 gene occur in diverse human tumor types,” Nature, 342:705708 (1989); Takahashi et al., “p53: A frequent target for genetic abnormalities in lung cancer,” Science, 246:491-194 (1989)). In addition, some studies suggest that individuals with inherited mutations of p53 are predisposed to a variety of cancers (Malkin et al., “Germ line p53 mutations in a familial syndrome of breast cancer, sarcomas, and other neoplasms,” Science, 250:1233-1238 (1990); Srivastava et al., “Germ-line transmission of a mutated p53 gene in a cancer-prone family with Li-Fraumeni syndrome,” Nature, 348:747-749 (1990); Li et al., “A cancer family syndrome in twenty-four kindreds,” Cancer Res, 48:5358-5362 (1988)). It has been shown that the tumors of these individuals have lost the wildtype p53 allele which is reminiscent of the loss of heterozygosity of the retinoblastoma tumor suppressor gene in retinoblastoma and other tumors (Knudson, A. G. “Mutation and Cancer: Statistical study of retinoblastoma,” Proc. Natl. Acad. Sci., USA, 68:820-823 (1971); Comings, D. E. “A general theory of carcinogenesis,” Proc. Natl. Acad. Sci., USA, 70:3324-3328 (1973)).

Some studies disclose that in vitro introduction of the wild-type p53 gene into a variety of different tumor lines results in down regulation of cell proliferation in culture or suppression of the tumorigenic phenotype upon reimplantation of the cells in vivo. These studies include tumor cells derived from glioblastomas (Mercer et al., “Negative growth regulation in a glioblastoma tumor cell line that conditionally expresses human wild-type p53,” Proc. Natl. Acad. Sci., USA, 87:6166-6170 (1990)), colon carcinoma (Baker et al., “Suppression of human colorectal carcinoma cell growth by wild-type p53,” Science, 249:912-915 (1990)), osteosarcoma (Diller et al., “p53 functions as a cell cycle control protein in osteosarcomas,” Mol. Cell. Biol., 10:5772-5781 (1990); Chen et al., “Genetic mechanisms of tumor suppression by the human p53 gene,” Science, 250:1576-1580 (1990)), leukemia (Cheng et al., “Suppression of acute lymphoblastic leukemia by the human wild-type p53 gene,” Cancer Res., 53:222-226 (1992)), and lung carcinoma (Takahashi et al., “Wild-type but not mutant p53 suppresses the growth of human lung cancer cells bearing multiple genetic lesions,” Cancer Res., 52:2340-2343 (1992)). However, in vitro introduction of the wild-type p53 gene into non-malignant cells does not result in the reduced cell growth as seen in tumor cell lines (Baker et al., supra).

Not all tumor cells with p53 mutations display significant down regulation of proliferation by wild-type p53 expression. Hinds et al. Cell Growth and Differentiation 1:571-80, (1990) disclosed that not all p53 mutants result in equivalent phenotypes. Michalovitz et al. Cell 62:671-680, (1990) disclosed that some mutants of p53 may be dominant to wild-type p53 with regard to growth regulation. Expression of wild-type p53 does not affect growth properties of some tumor cell lines, including human papillomavirus-expressing cell lines, and A673 rhabdomyosarcoma cells (Chen et al., Oncogene 6:1799-1805, 1991). In those cases where p53 was reported to suppress cell proliferation, the effect was sometimes small (Cheng et al., 1992, supra).

Furthermore, the method of using wild-type p53 alone to down-regulate tumor cells requires stable wild-type p53 expression in tumor cells. In studies of a temperature sensitive mutant of p53, it has been observed that the suppressive effect of wild-type p53 on the proliferation of transformed cells was lost when wild-type p53 expression ceased (Michalovitz et al., 1990, supra). Since the most efficient gene transfer approaches presently available provide only transient expression of p53, this limits the efficacy of therapy with p53 alone.

p53 function is highly complex and has been implicated in a variety of cellular processes including proliferation (Baker et al., Science 249: 912-915,1990; Michalovitz et al., Cell 62: 671-680, 1990), differentiation (Shaulsky et al., Proc. Natl. Acad. Sci. 88: 8982-8986, 1991), programmed cell death (i.e., apoptosis)(Yonish-Rouach et al., Nature 352: 345-347, 1991), cellular senescence (Shay et al., Exp. Cell Research. 196: 33-39, 1991), DNA binding (Kern et al., Science 252: 1708-1711, 1991; Bargonetti et al., Cell 65: 1083-1091, 1991), and DNA damage-induced G1 arrest (Kastan et al., Cancer Research 51: 6304-6311, 1991; Kuerbitz et al., Proc. Natl. Acad. Sci. USA 89: 7491-7495, 1992). With regard to cancer therapy, the involvement of p53 in DNA damage-induced G1 arrest is one of its most provocative roles.

Wild-type and mutant p53 genes have been transferred into tumor cells lacking endogenous p53. When these cells were exposed to gamma irradiation, the expression of wild-type p53 led to transient cell cycle arrest at the G1/S phase boundary (Kastan et al., “Participation of p53 protein in the cellular response to DNA damage,” Cancer Res., 51:6304-6311 (1991); Kuerbitz et al., “Wild-type p53 is a cell cycle checkpoint determinant following irradiation,” Proc. Natl. Acad. Sci., USA, 89:7491-7495 (1992); Yonish-Rouach et al., “Wild-type p53 induces apoptosis of myeloid leukaemic cells that is inhibited by interleukin-6,” Nature, 352:345-347 (1991)). Cells which lacked p53 or which expressed mutant p53 did not arrest (Kastan et al., supra; Kuerbitz et al., supra).

It has been proposed that p53 plays an important checkpoint function by preventing entry into S phase until DNA damage is repaired (Vogelstein et al., Cell 70: 523-526, 1992). Thus, the outcome of DNA damaging radiation or chemotherapy on cancer cells may be affected by the expression of mutant or wild-type p53.

In this regard, several lines of evidence suggest that cancer cells which have lost wild-type p53 function are more sensitive to DNA damaging drugs and radiation. By analogy to similar checkpoints in yeast, failure of p53 induced G1 arrest could enhance cell destruction by preventing repair of potentially lethal DNA damage prior to cell division (Vogelstein et al., supra).

Vogelstein et al., supra, stated:

tumor cells are often more sensitive to DNA-damaging agents such as those used in radiation and chemotherapy; this sensitivity may be a beneficial side effect of the loss of p53 function, which would otherwise limit cell death. p53 mutations may therefore constitute one of the few oncogenic alterations that increase rather than decrease the sensitivity of cells to antitumor agents.

This view is supported by studies demonstrating increased sensitivity of tumor cells to radiation and chemotherapy following mutated p53 gene transfer (Petty et al., “Expression of the p53 tumor suppressor gene product is a determinant of chemosensitivity,” Biochem. Biophys. Res. Comm. 199:264-270, 1994, not admitted to be prior art).

However, other studies performed with normal hematopoietic cells, fibroblasts, and gastrointestinal cells from p53 null transgenic mice indicated a requirement for p53 in apoptosis (Lowe et al., Nature 362: 847-849, 1993; Clarke et al., Nature 362: 849-852, 1993; Lotem J and Sachs L, Blood 82: 1092-1096, 1993; Lowe et al., Cell 74: 957-967, 1993; and Merritt et al., Cancer Research 54:614-617, 1994, not admitted to be prior art). In these studies, normal cells lacking p53 were more resistant to apoptosis following exposure to radiation or DNA damaging drugs. Similarly, a study of Burkitt's lymphoma cell lines revealed that some but not all cell lines with wild-type p53 gene configurations were more sensitive to radiation (O'Connor et al., Cancer Research 53: 4776-4780, 1993, not admitted to be prior art). However, evaluation of head and neck cancer cell lines showed no correlation between radiation sensitivity and expression of either endogenous wild-type or mutant p53 (Brachman et al., Cancer Research 53: 3667-3669, 1993, not admitted to be prior art).

Lowe, et al., “p53-dependent apoptosis modulates the cytotoxicity of anticancer agents,” Cell, 74:957-967, 1993 (not admitted to be prior art) stated:

p53-deficient mouse embryonic fibroblasts were used to examine systematically the requirement for p53 in cellular sensitivity and resistance to a diverse group of anticancer agents. These results demonstrate that an oncogene, specifically the adenovirus E1A gene, can sensitize fibroblasts to apoptosis induced by ionizing radiation, 5-fluorouracil, etoposide, and adriamycin. Furthermore, the p53 tumor suppressor is required for efficient execution of the death program.

Lotem and Sachs, “Hematopoietic cells from mice deficient in wild-type p53 are more resistant to induction of apoptosis by some agents,” Blood, 82:1092-1096 (1993) (not admitted to be prior art) stated:

In normal fibroblasts, irradiation and other DNA-damaging agents induce the expression of wild-type p53 and this induction of wild-type p53 arrests cells at a control point in G1. It was suggested that this G1 arrest is required for DNA repair before the onset of DNA replication to prevent the propagation of DNA damage. Fibroblasts from p53-deficient mice lost this G1 control, continued the cell cycle after irradiation, and thus propagated the DNA damage. our results show that, under conditions of high concentration of viability factors, there was no difference in the number of myeloid colony-forming cells in mice with or without wild-type p53. However, when myeloid progenitor cells had only a low concentration of viability factors such as GM-CSF, IL-1α, IL-3, IL-6, or SCF, or when apoptosis was induced in these cells by irradiation or heat shock, cells from p53-deficient mice had a higher viability. The comparison of mice homozygous and heterozygous for p53 deficiency showed that the loss of one allele of wild-type p53 was sufficient for increased resistance to the induction of apoptosis. The higher resistance to induction of apoptosis in p53-deficient mice was also found in irradiated thymocytes, but not in thymocytes treated with the glucocorticoid dexamethasone or in mature peritoneal granulocytes. The degree of resistance in irradiated myeloid progenitors and thymocytes was related to the dose of wild-type p53.

Hence, the effects of mutant and wild type p53 on chemotherapy and radiation sensitivity are unclear from these previous investigations and none of these earlier studies addressed the effects of wild-type p53 gene transfer on treatment sensitivity in tumor cells expressing endogenous mutant p53.

Enhancing the Effect of a Cancer Therapy

This invention features a new method for enhancing the effect of a cancer therapy by introducing into tumor cells a source of wild-type therapy-sensitizing gene activity before subjecting the tumor cells to therapy. Using p53 as an example of therapy-sensitizing gene, this invention can be carried out as follows:

First, a patient's tumor is determined to contain a p53 mutation by standard diagnostic methods. Wild-type p53 activity such as a portion of p53 protein having therapy-sensitizing activity or a gene expression vector encoding said portion of p53 protein is then introduced into the tumor cells. This renders the tumor cells with the p53 mutation more sensitive to a cancer therapy administered during the period of wild-type p53 activity. The cancer therapies whose effect may be enhanced by this method include, but are not limited to, radiotherapy, chemotherapy, biological therapy such as immunotherapy, cryotherapy and hyperthermia.

In a cell with mutated therapy-sensitizing gene activity such-as mutant p53 protein, unrepaired DNA damage may not block entry into S phase or trigger apoptosis. Without being bound by any theory, applicant believes that tumor cells which have endogenous mutant therapy-sensitizing gene activity and which have been restored with wild-type therapy-sensitizing activity such as wild-type p53 gene or protein would be particularly sensitive to induction of apoptosis by therapeutic modalities given the intrinsic susceptibility of tumor cells to genomic damage and an overloaded or impaired repair process. The presence of wild-type therapy-sensitizing gene activity in the tumor cells would sensitize such cells to these DNA damaging agents, and probably also to a variety of other therapeutic modalities which may induce apoptosis.

The method of combining p53 sensitization therapy with other therapy is more effective than either therapy alone. When exogenous wild-type p53 activity is introduced into a tumor cell, lower doses of drugs or radiation are needed to kill the cell, and the therapeutic window of concentrations over which drugs or radiation can be administered without toxicity is increased. In contrast to p53 gene therapy-alone, which requires sustained p53 gene expression for tumor suppression, the combined effects of p53 sensitization therapy with other treatments requires only transient existence of a therapy-sensitizing portion of a wild-type p53 protein in the tumor cell during the treatment period to kill the tumor cell. This method also improves the efficacy of biological therapies, including, but not limited to, immunotherapies, such as passive immunotherapies (e.g., antibodies); adoptive immunotherapies involving the administration of activated immune system effector cells; active immunotherapies involving immunization to induce antitumor immunity; therapies mediated by various cytokines, including, but not limited to, interleukins such as IL-2, IL6, IL-7, IL-12, tumor necrosis factors, tumor growth factors, interferons, growth factors such as GM-CSF and G-CSF by increasing tumor cells' sensitivity to these cytokines or to the effector mechanisms of the immune system activated by these cytokines. Furthermore, the claimed p53-mediated sensitization therapy makes tumor cells better targets for the immune system by restoring the apoptotic pathways required for killing by cytotoxic immune cells, including, but not limited to, cytotoxic T cells, lymphokine activated killer cells, natural killer cells, macrophages, monocytes, and granulocytes.

The therapy-sensitizing activity may be embodied in a portion or portions of wild-type p53 gene/protein. A therapy-sensitizing portion may be delineated by routine mutation analysis, such as point mutations and deletion mutations, known to those skilled in the art.

Small molecules which mimic the wild-type therapy-sensitizing gene product activity may also be employed to enhance cancer therapy, including, but not limited to, peptides, modified peptides or organic chemical compounds. Other useful agents include small molecules which bind to mutated therapy-sensitizing gene products and serve as allosteric regulators inducing a conformational change which establishes the wild-type therapy-sensitizing activity of that gene product.

Because p53 or other therapy-sensitizing gene mutations have been observed in virtually every cancer examined, this invention has very broad application. In a preferred embodiment, tumors that are localized can be treated by direct delivery of a portion of the wild-type p53 gene encoding the therapy-sensitizing activity to the tumor cells, using presently available gene delivery vehicles, including, but not limited to, infection by p53 adenovirus vector, implantation of a p53 retrovirus vector packaging line, or transfection of p53 cDNA facilitated by adenovirus capsids in a linked complex. With the development of targeting approaches which permit accumulation of gene transfer vectors at the tumor site, this approach can be extended to disseminated cancers. Other gene-expression vector systems may also be utilized, including, but not limited to, lipofection or direct DNA injection. Other methods of gene transfer and expression known to those skilled in the art may also be utilized. The examples provided below for the therapy sensitizing gene p53 may also be adapted by one skilled in the art to other therapy-sensitizing genes for the treatment of cancer.

### EXAMPLE 1.

### Transferring a P53 Gene into a Tumor Cell

The wild-type p53 gene or a part of the gene may be introduced into a tumor cell in a vector, such that the gene remains extrachromosomal. Wild-type p53 protein is expressed from the extrachromosomal wild-type p53 gene or a part of the gene.

Alternatively, the wild-type p53 gene may be introduced into a tumor cell in such a way that it replaces the endogenous mutant p53 gene present in the cell. This approach would result in the correction of the p53 gene mutation (Revet et al., “Homologous DNA targeting with RecA protein-coated short DNA probes and electron microscope mapping on linear duplex molecules,” Journal of Molecular Biology, 232(3):779-91, 1993; Thomas et al., “High-fidelity gene targeting in embryonic stem cells by using sequence replacement vectors,” Molecular and Cellular Biology, 12(7):2919-23, 1992; Mansour et al., “Introduction of a lacZ reporter gene into the mouse int-2 locus by homologous recombination,” Proc. Natl. Acad. Sci. 87(19):7688-92, 1990; Capecchi, “Altering the genome by homologous recombination,” Science, 244(4910):1288-92, 1989; Sedivy and Joyner, “Gene targeting,” published by W. H. Freeman, 1992; incorporated by reference herein).

A preferred vector for p53 gene transfer has the ability to transfer the gene to all or most of the cells in the target cell population, and to achieve sufficiently long expression and sufficiently high expression levels to promote the desired effect. Possible vector designs and gene transfer approaches include but are not limited to the following:

1). Adenovirus vectors. Adenoviral vectors can be obtained in higher titer than retroviral vectors, enabling a potentially higher efficiency of gene delivery. They are particularly attractive in being able to infect a broad range of cell types, both dividing and non-dividing (Graham FL, Prevec L. Manipulation of adenovirus vectors. In Murray E J, ed. “Methods in Molecular Biology” vol. 7, Gene Transfer and Expression Protocols” Clifton, N.J.; The Humana Press, Inc. (1991). pp. 109-128, incorporated by reference herein). These vectors replace part of the early region gene required for viral replication with the transgene (i.e., an exogenous gene to be transferred to a cell) of interest. Virus particles are obtained by transfecting the DNA into an appropriate packaging cell line which supplies the missing replication functions. Examples of such vectors have been described (Berner K L. “Development of Adenovirus vectors for the expression of heterologous genes,” Biotechniques. (1988) 6:6616-629, incorporated by reference herein). Intra-arterial infusion of adenovirus vectors would be suitable for, but not limited to, liver cancer and head and neck cancers.

2). Retroviral vectors. These vectors are the best characterized for human gene transfer, and have been used in gene therapy protocols (Wu et al., J. of Biochemistry, 266:14338-14342, 1991, incorporated by reference herein). Retroviral vectors consist of a modified retroviral genome containing the gene of interest to be transferred (i.e. transgene), and often a selectable marker gene. The vector itself provides the viral LTR (Long Terminal Repeat) sequences necessary for stable integration of the gene, but is defective for replication and requires a packaging cell line to provide the transacting replication factors. Examples of retroviral vectors and packaging cell lines have been described (Kriegler, M. (1990) “Gene Transfer and Expression—A Laboratory Manual,” Stockton Press, New York, and Jolly, D., Cancer Gene Therapy, 1:51-64, 1994, incorporated by reference herein). A p53-retroviral vector has been described (Cheng et al., (1992) “Suppression of acute lyphoblastic leukemia by the human wild-type p53 gene,” Cancer Res. 52:222-226, incorporated by reference herein).

Retroviral vectors have a broad range of infectivity with respect to cell type. Transgene expression is usually driven from a strong viral promoter which has broad tissue specificity. Examples are the viral LTR (Long Terminal Repeat), Cytomegalovirus (CMV) promoter, Simian virus 40 (SV40) promoter (Miller A D and Rosman G J. “Improved retroviral vectors for gene transfer and expression,” (1989) BioTechniques 7:980-990, incorporated by reference herein).

A packaging cell line secreting the p53 retroviral vector can be implanted at the tumor site to increase the efficiency of retroviral gene transfer. The cell line provides a continuous source of vector and improves the efficiency of gene transfer (Culver K W, Ram Z, Wallbridge S, Ishii H, Oldfield E H, Blaese R M. In vivo gene transfer with retroviral vector-producer cells for treatment of experimental brain tumors. (1992) Science 256: 1550-2).

3). Adeno-associated virus. Vectors based on adeno-associated virus have the range of infectability of adenovirus. In addition, these vectors provide the potential for stable integration of exogenous DNA at preferred sites in the host genome. A discussion of such vectors can be found in “Current Topics in microbiology and Immunology” vol. 158, (Muzyczka N, ed), Springer-Verlag, pp. 97-129, (1992), herein incorporated by reference.

4). Other viral vectors. Vectors based on herpes, vaccinia, papilloma virus can also be used to transfer gene to tumor cells. A discussion of these vectors can be found in Kriegler, M. “Gene Transfer and Expression. A Laboratory Manual.” Stockton Press, New York, (1990); and Jolly D. “Viral Vectors for Gene Therapy,” Cancer Gene Therapy vol. 1:51-64 (1994), herein incorporated by reference.

5). Coupled adenovirus capsids. Exogenous DNA may be transferred to a tumor cell by an adenovirus capsid. In this approach, the DNA to be transferred is coupled to the outside of the virus capsid through a polylysine bridge (Curiel, et al., “High efficiency gene transfer mediated by adenovirus coupled to DNA-polylysine complexes,” (1992) Human Gene Therapy, 3:147-154, incorporated by reference herein). Entry of DNA into the cell is achieved through the natural pathways of virus internalization, but gene transfer and expression is independent of the viral genome. For example, p53 gene can be coupled to an adenovirus capsid which in turn is delivered into a lung carcinoma cell by receptor-mediated endocytosis. Thus in this approach the virus particle is used as a carrier for transfection of DNA rather than as a vehicle for infection. High efficiencies of gene transfer can be achieved with this approach, particularly when the complex of virus and DNA incorporates an additional ligand such as but not limited to transferrin (Wagner et al., “Coupling of adenovirus to transferrin-polylysine/DNA complexes greatly enhances receptor-mediated gene delivery and expression of transfected genes,” (1992) Proc. Natl. Acad. Sci., USA, 89:6099-6103, incorporated by reference herein). Tissue and cell type specific ligands can also be incorporated to facilitate accumulation of the complex in the target tissue.

6). Other methods. Liposome-mediated gene transfer is effective for in vivo gene delivery (Zhu et al., “Systemic gene expression after intravenous DNA delivery into adult mice,” (1993) Science 261:209-11; Yoshimura et al., (1992) Nucleic Acids Research 20:3233-3240; incorporated by reference herein). A DNA-liposome complex can be administered locally or systemically. The advantage of this approach is low toxicity and absence of viral genomes. With the choice of an appropriate promoter (e.g., CMV promoter), an extended period of expression can be achieved (Zhu et al., “Systemic gene expression after intravenous DNA delivery into adult mice,” (1993) Science 261: 209-11, incorporated by reference herein).

In addition, ligand-DNA conjugates have been utilized to target transgene-expression to specific cell types. For example, asialoglycoprotein-DNA conjugates have been used to target exogenous genes specifically to hepatocytes via the asialoglycoprotein receptor. Direct gene transfer of naked DNA may be effective for some tissues as well, such as, but not limited to, muscle. These methods of gene transfer may be applied singly or in combination by those skilled in the art to achieve the expression in the tumor of a portion of a wild-type p53 gene or other therapy-sensitizing gene encoding the therapy-sensitizing activity.

### EXAMPLE 2.

### Introduction of p53 Protein to a Tumor Cell

Wild-type p53 protein or a portion of the wild-type p53 protein which has therapy-sensitizing activity may be supplied to cells which carry mutant p53 alleles. This may be achieved in vivo by several methods including but not limited to intravenous, intra-tumoral, intra-arterial, intra-cavitary, or intrathecal infusions. Aerosolized preparations may be employed for delivery to the respiratory tract and topical preparations may also be utilized. The active molecules can also be introduced into the cells by microinjection, by liposomes, or by electroporation methods. The p53 protein can also be introduced into tumor cells by receptor-mediated endocytosis. Alternatively, p53 protein may be actively taken up by the cells, or taken up by diffusion, to restore p53 activity to the cells.

A chimeric protein comprising p53 and a targeting sequence can be used to introduce wild type p53 activity into a cell bearing a receptor for the targeting sequence. For example, the targeting specificity of insulin-like-growth-factor-I (IGF-I) or Interleukin-2 (IL-2) can be used to deliver p53 protein to IGF-I receptor or IL-2 receptor bearing cells. The chimeric protein can be obtained by constructing chimeric cDNAs through recombinant techniques and expressing them in either procaryotic or eucaryotic systems.

Thus, when p53 is chimerized to growth factor IGF-I, which binds to specific cell surface receptors on lung carcinoma cells, the chimeric protein can be targeted to lung carcinoma cells by receptor mediated endocytosis.

### EXAMPLE 3.

### Administration of Agents

In practicing the methods of the invention, the compositions, such as those discussed in Examples 1 and 2 above, can be used alone or in combination with one another, or in combination with other therapeutic or diagnostic agents. These compositions can be utilized in vivo to a human patient, or in vitro. In employing them in vivo, the compositions can be administered to the patient in a variety of ways, including but not limited to parenterally, intravenously, subcutaneously, intramuscularly, colonically, rectally, vaginally, nasally, orally, transdermally, topically, ocularly, intraperitoneally, intracavitarily, intrathecally or as suitably formulated surgical implants employing a variety of dosage forms.

The dosage for the compositions of the present invention can range broadly depending upon the desired effects and the therapeutic indication. As will be readily apparent to one skilled in the art, the useful in vivo dosage to be administered and the particular mode of administration will vary depending upon, the condition of the patient, the cancer treated and the particular composition employed. The determination of effective dosage levels, i.e. the dosage levels necessary to achieve the desired result, will be within the ambit of one skilled in the art. Typically, applications of compositions are commenced at lower dosage levels, with dosage level being increased until the desired effect is achieved.

Effective delivery requires the agent to enter into the tumor cells. Chemical modification of the agent may be all that is required for penetration. However, in the event that such modification is insufficient, the modified agent can be co-formulated with permeability enhancers, such as but not limited to Azone or oleic acid, in a liposome. The liposomes can either represent a slow release presentation vehicle in which the modified agent and permeability enhancer transfer from the liposome into the transfected cell, or the liposome phospholipids can participate directly with the modified agent and permeability enhancer in facilitating cellular delivery.

Drug delivery vehicles may be employed for systemic or topical administration. Topical administration of agents is advantageous since it allows localized concentration at the site of administration with minimal systemic absorption. This simplifies the delivery strategy of the agent to the disease site and reduces the extent of toxicological characterization. Furthermore, the amount of material to be administered is far less than that required for other administration routes.

Agents may also be systemically administered. Systemic absorption refers to the accumulation of drugs in the blood stream followed by distribution throughout the entire body. Administration routes which lead to systemic absorption include but are not limited to: oral, intravenous, intraarterial, intralymphtic, subcutaneous, intraperitoneal, intranasal, intramuscular, intrathecal and ocular. Each of these administration routes exposes the agent to an accessible diseased tissue. Subcutaneous administration drains into a localized lymph node which proceeds through the lymphatic network into the circulation. The rate of entry into the circulation has been shown to be a function of molecular weight or size. Intraperitoneal administration may also lead to entry into the circulation with the molecular weight or size of the agent-delivery vehicle complex controlling the rate of entry.

Drug delivery vehicles can be designed to serve as a slow release reservoir, or to deliver their contents directly to the target cell. An advantage of using direct delivery drug vehicles is that multiple molecules are delivered per vehicle uptake event. Such vehicles have been shown to also increase the circulation half-life of drugs which would otherwise be rapidly cleared from the blood stream. Some examples of such specialized drug delivery vehicles which fall into this category include but are not limited to liposomes, hydrogels, cyclodextrins, biodegradable polymers (surgical implants or nanocapsules), and bioadhesive microspheres.

Liposomes offer several advantages: They are generally non-toxic and biodegradable in composition; they may display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

Orally-administered formulations can be prepared in several forms, including but not limited to capsules, chewable tablets, enteric-coated tablets, syrups, emulsions, suspensions, or as solid forms suitable for solution or suspension in liquid prior to administration. Suitable excipients are, for example, water, saline, dextrose, mannitol, lactose, lecithin, albumin, sodium glutamate, cysteine hydrochloride or the like. In addition, if desired, the pharmaceutical compositions may contain minor amounts of nontoxic auxiliary substances, such as wetting agents, pH buffering agents, and the like. If desired, absorption enhancing preparations (e.g., liposomes) may be utilized.

### EXAMPLE 4.

### Increasing Tumor Cells' Sensitivity to Chemotherapy

1). Cells. T98G glioblastoma cells (Mercer et al., “Negative growth regulation in a glioblastoma tumor cell line that conditionally expresses human wild-type p53.” (1990) Proc. Natl. Acad. Sci., USA, 87:6166-6170) were obtained from ATCC and cultured at 37° C. in 10% CO₂ in Dulbecco's Modified Eagles Medium supplemented with 10% heat inactivated fetal bovine serum, gentamycin, nonessential amino acids, and sodium pyruvate. These cells are derived from a biopsy of a patient with glioblastoma mulitforme and have been shown to have a homozygous mutation in the p53 gene at codon 237 (from met to ile, ATG to ATA) (Ullrich et al., “Human wild-type p53 adopts a unique conformational and phosphorylation state in vivo during growth arrest of glioblastoma cells.” (1992) Oncogene, 7(8):1635-43).

2). Plasmids. A plasmid (pLp53RNL) containing the wild-type p53 gene and the neomycin (G418) resistance gene was used. The plasmid pLp53RNL was kindly provided by Dr. Martin Haas (University of California, San Diego), and has been previously described (Cheng et al., “Suppression of acute lymphoblastic leukemia by the human wild-type p53 gene,” (1992) Cancer Res., 53:222-226). This plasmid carries the retroviral sequence Lp53RNL in which wild-type p53 expression is driven from the Moloney murine leukemia virus (MoMLV) LTR. The neomycin resistance gene is driven from the Rous Sarcoma Virus (RSV) promoter.

3). Transfections. The plasmid was introduced into T98G cells using cationic liposomes. T98G cells were plated in 10 cm culture dishes at about 5×10 ⁵ cells per plate. The following day cells were transfected with 15 μg DNA using Lipofectamine (BRL) and following the manufacturer's instructions. Five days following transfection, cultures were selected in 100 μg/ml G418. Clones were picked about three weeks later and expanded. Prior to determining growth kinetics and plating efficiencies, cultures were adapted to growth in the absence of G418 for 7-10 days. One colony is denoted T98Gp53because it contains the exogenous wild-type p53 gene.

4). Plating efficiency. Cells were plated in triplicate at low density, 100-500 cells per 6 cm plate, and allowed to grow for two weeks. Plates were stained in 0.5% methylene blue in methanol and colonies were counted. Plating efficiency of transfected cells was 20%. Parental cells had a plating efficiency of 50%.

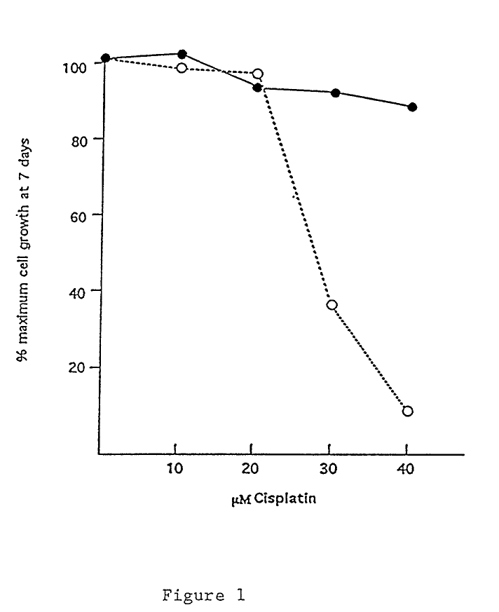

5). Control parental T98G cells and T98Gp53 cells which had been adapted 2 weeks to culture in the absence of the antibiotics G418 were plated in 24 well plates at about 2×10⁴ cells per well. The next day they were exposed for one hour to varying concentrations of cisplatin (a chemotherapeutic agent) from 10 to 40 μM in increments of 10 μM. The cisplatin was removed after one hour and replaced with complete medium (DMEM+10% Fetal Bovine Serum) and cells were allowed to grow for 7 days. After 7 days, cells were counted or stained with crystal violet. In the latter case, absorbance at 540 nm is proportional to cell viability. For clonogenic assays, cells were replated following treatment in 6 well plates at 500-1000 cells per well. Clones were counted 7 to 10 days later by staining in 0.5% methylene blue, 70% ETOH. Colony counts from p53 transfectants and parental T98G glioblastoma cells were compared. As shown in FIG. 1, T98Gp53 cells were considerably more sensitive to the effects of cisplatin than were the parental T98G cells. Subsequent assays confirmed this increased sensitivity. The concentration of cisplatin needed to achieve a 50% reduction in colony count was reduced from about 30 μM in the case of T98G parental cells and empty vector-transduced cells to 15-20 μM cisplatin in the case of cells transduced with wild-type p53 gene.

### EXAMPLE 5.

### Increasing Tumor Cells' Sensitivity to Radiotherapy

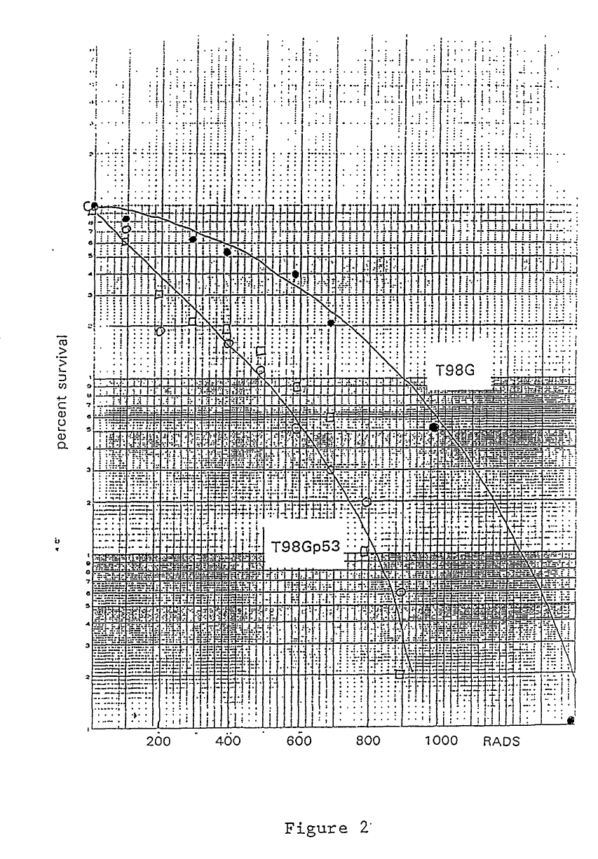

Control parental T98G cells and T98Gp53 cells were grown for two weeks without G418 and then plated at about 5,000 cells per T25 flask. The next day, cells were subjected to gamma radiation from a Cobalt 60 source in doses ranging from 100 rads to 1500 rads in increments of 100 rads. Cells were then incubated for an additional 5-12 days and colonies were stained in 0.5% methylene blue-methanol, counted, and compared to control untreated cells. As shown in FIG. 2, wild-type p53 transduced T98Gp53 cells show enhanced sensitivity to radiation, with 50% reduction in colony counts occurring at about 200 rads as compared to 400 rads for the parental cells.

### EXAMPLE 6.

### P53 Gene Sensitization Therapy

The treatment described below applies to tumors with mutant p53 activity.

1. Identification of Tumors with P53 Abnormalities

Routine molecular biology diagnostic techniques can be used to identify tumors that have p53 abnormalities, including, but not limited to, single-strand conformation polymorphism (SSCP), PCR, sequencing and related molecular biology methods to detect gene abnormalities known to those skilled in the art (“General Molecular Biology Methods—Current Protocols in Molecular Biology,” John Wiley and Sons, 1994; and J. Sambrook, E. F. Fritsch, and T. Maniatis, Molecular Cloning: A Laboratory Manual, 2 Ed., Cold Spring Harbor Laboratory Press, Cold Spring Harbor, New York, 1989, incorporated by reference herein).

2. Sensitization of Tumors with P53 Vectors By Direct Injection or Aerosolized Preparations

In this application, a suitable wild-type p53 vector and/or producer cell line is injected into a tumor or into a former tumor site following surgical resection or ablation (to treat residual tumor cells) to permit expression by the tumor cell of a portion of a wild-type p53 gene encoding the therapy-sensitizing activity. Aerosolized vector preparations may also be utilized to deliver wild-type p53 to resection sites or tumors in the respiratory tract. Subsequently, the patient is treated with chemotherapy, radiotherapy, biological therapy, cryotherapy or hyperthermia appropriate for the treatment of said tumor known to those skilled in the art as described in “Cancer:Principles and Practice of Oncology,” Devita, Hellman, Rosenberg Eds., Lippencott, 1993; “Manual of Oncologic Therapeutics,” Wittes Ed., Lippencott, 1993; and “Biologic Therapy of Cancer,” Devita et al., eds., Lippencott, 1991, incorporated by reference herein.

This approach may be employed to treat localized primary tumors including but not limited to central nervous system tumors, sarcomas, and early stage carcinomas (lung, prostate, breast, bladder, kidney, hepatocellular, pancreatic, gastric, esophageal, colorectal, anal, head and neck, biliary, and urogenital).

This approach may also be utilized to treat metastatic lesions of these and other tumors. In these applications, a suitable wild-type p53 vector and/or producer cell line is injected into a metastatic tumor or into the metastatic tumor site following surgical resection or ablation to permit expression by the tumor cell of a portion of a wild-type p53 gene encoding the therapy-sensitizing activity. Aerosolized vector preparations may also be utilized to deliver wild-type p53 to resection sites or tumors in the respiratory tract. Subsequently, the patient is treated with chemotherapy, radiotherapy, biological therapy, cryotherapy, or hyperthermia appropriate for the treatment of said metastatic tumor known to those skilled in the art as described in “Cancer:Principles and Practice of Oncology,” Devita, Hellman, Rosenberg Eds., Lippencott, 1993; and Manual of Oncologic Therapeutics, Wittes Ed., Lippencott; and “Biologic Therapy of Cancer,” Devita et al., eds., Lippencott, 1991, incorporated by reference herein.

3. Sensitization of Tumors with p53 Vectors by Intra-Arterial Infusion

Intra-arterial infusion chemotherapeutic drugs and other agents has been utilized in the treatment of numerous forms of primary and metastatic cancers (Cancer:Principles and Practice of Oncology, Devita, Hellman, Rosenberg Eds., Lippencott; and Manual of Oncologic Therapeutics, Wittes Ed., Lippencott.). In this application of p53 therapy-sensitization, these intra-arterial infusion methods are employed to deliver a suitable wild-type p53 vector and/or producer cell line to permit expression by the tumor cell of a portion of the wild-type p53 gene encoding the therapy-sensitizing activity. Subsequently, the patient is treated with chemotherapy, radiotherapy, biological therapy, cryotherapy or hyperthermia appropriate for the treatment of said primary or metastatic tumor known to those skilled in the art as described in Cancer:Principles and Practice of Oncology, Devita, Hellman, Rosenberg Eds., Lippencott; and Manual of Oncologic Therapeutics, Wittes Ed., Lippencott; and “Biologic Therapy of Cancer,” Devita et al., eds., Lippencott, 1991, incorporated by reference herein. This approach may be applied to the treatment of tumors such as but not limited to primary hepatocellular carcinoma, liver metastases and head and neck tumors. This approach may be adapted by those skilled in the art of arterial infusion to treat any tumor with an accessible arterial vasculature for infusion.

4. Sensitization of Tumors with p53 Vectors by Intracavitary Infusion

In these applications, body cavities containing tumor cells are first infused with a suitable wild-type p53 vector and/or producer cell line to permit expression by the tumor cells of a portion of a wild-type p53 gene encoding the therapy-sensitizing activity. Subsequently, the patient is treated with chemotherapy, radiotherapy, biological therapy, cryotherapy, or hyperthermia appropriate for the treatment of said primary or metastatic cavitary tumor known to those skilled in the art as described in Cancer:Principles and Practice of oncology, Devita, Hellman, Rosenberg Eds., Lippencott; and Manual of Oncologic Therapeutics, Wittes Ed., Lippencott; and “Biologic Therapy of Cancer,” Devita et al., eds., Lippencott, 1991, incorporated by reference herein.

This approach may be applied but is not limited to the treatment of malignant pleural effusions (pleural cavity), ascites (abdominal/peritoneal cavity), leptomeningeal tumors (cerebrospinal/ventricular system), pericardial effusions (pericardial cavity) and bladder carcinomas (bladder infusions).

5. Tumor Purging of Hematopoietic Stem/Progenitor Cells by P53 Sensitization

In this application, autologous hematopoietic stem/progenitor cells are purged of residual tumor cells by p53 sensitization before they are utilized to rescue patients from the effects of myelosuppressive/ablative cancer therapies. Hematopoietic stem/progenitor cell preparations are harvested from the patient by standard methods (Cancer:Principles and Practice of Oncology, Devita, Hellman, Rosenberg Eds., Lippencott; and Manual of Oncologic Therapeutics, Wittes Ed., Lippencott; and “Bone Marrow Transplantation,” Forman et al. Eds., 1993, incorporated by reference herein) and transduced ex vivo with a suitable wild-type p53 vector and/or producer cell line to permit expression of a portion of a wild-type p53 gene encoding the therapy-sensitizing activity. Subsequently, the transduced cell preparation is subjected to cytotoxic purging techniques known to those skilled in the art (Cancer:Principles and Practice of Oncology, Devita, Hellman, Rosenberg Eds., Lippencott; and Manual of Oncologic Therapeutics, Wittes Ed., Lippencott; and “Bone Marrow Transplantation,” Forman et al. Eds., 1993, incorporated by reference herein).

The patients are then treated with myelosuppressive/ablative cancer therapy and the hematopoietic stem-progenitor cells purged of residual tumor cells by p53 sensitization are then infused into patients to rescue them from the myelosuppressive effects of very aggressive cancer treatment.

The administration of myelosuppressive/ablative treatment and rescue by hematopoietic stem/progenitor cell infusion is well described in the prior art and has been utilized to treat a wide variety of solid and hematopoietic malignancies (Cancer:Principles and Practice of Oncology, Devita, Hellman, Rosenberg Eds., Lippencott; and Manual of Oncologic Therapeutics, Wittes Ed., Lippencott; and “Bone Marrow Transplantation,” Forman et al. Eds., 1993, incorporated by reference herein). The p53 sensitization of residual tumor cells to destruction by cytotoxic purging agents will decrease the number of tumor cells in the hematopoietic stem-progenitor cell infusion utilized to rescue patients. This will decrease the likelihood of tumor recurrence which may occur from the infusion of hematopoietic stem/progenitor cell preparations which contain residual tumor cells.

6. Treatment of Disseminated Metastatic Tumor by p53 Sensitization

In this application, a suitable wild-type p53 vector and/or producer cell line is injected systemically or parenterally to permit expression by tumor cells of a portion of a wild-type p53 gene encoding the therapy-sensitizing activity. Subsequently, the patient is treated with chemotherapy, radiotherapy, biological therapy, cryotherapy or hyperthermia appropriate for the treatment of the metastatic tumor known to those skilled in the art as described in Cancer:Principles and Practice of Oncology, Devita, Hellman, Rosenberg Eds., Lippencott; and Manual of Oncologic Therapeutics, Wittes Ed., Lippencott; and “Biologic Therapy of Cancer,” Devita et al., eds., Lippencott, 1991, incorporated by reference herein.

The individual applications of p53-mediated sensitization therapy outlined above may also be utilized in combinations that may be applied by those skilled in the art of multimodality cancer therapeutics, for example, as described in Cancer:Principles and Practice of Oncology, Devita, Hellman, Rosenberg Eds., Lippencott; and Manual of Oncologic Therapeutics, Wittes Ed., Lippencott; and “Biologic Therapy of Cancer,” Devita et al., eds., Lippencott, 1991, incorporated by reference herein.

7. Treatment of Glioblastoma Multiforme by p53-mediated Sensitization Therapy

Glioblastoma multiforme represents the most frequently encountered intracranial brain tumor, with some 20,000 new cases being diagnosed each year in the U.S. Although it rarely metastasizes outside of the central nervous system, it is nevertheless the most malignant form of astrocytoma, and presents a therapeutic challenge to the physician employing present conventional approaches. These approaches include surgery, radiation, and chemotherapy, and while advances have been made in all areas, mean survival time from diagnosis is still only about one year. Glioblastomas are relatively radiation resistant, and respond poorly to most chemotherapeutic drugs. Of those chemotherapeutic agents which have been shown to have some effectiveness initially, including cisplatin, BCNU (carmustine) and PCV (procarbazine CCNU, vincristine), none shows sustained effectiveness.

Due to their location in the brain, the morbidity of even modest tumor progression in glioblastoma patients is high. Small decreases in tumor volume are expected to have a beneficial effect to patients. Furthermore, glioblastoma rarely metastasizes outside the central nervous system, making this disease an ideal target for localized gene transfer, including local infection with p53 bearing adenovirus, or local transfection with p53 cDNA facilitated by adenovirus capsids, or implantation of a p53 bearing viral vector packaging line at the tumor site. Similarly, this approach could have benefit for brain metastases of other cancers in which a decrease in morbidity may result from even small reductions in tumor volumes.

8. Treatment of Hepatocellular Carcinoma and Head and Neck Cancers by p53-mediated Sensitization Therapy

Hepatocellular carcinoma and head and neck cancers are characterized by frequent p53 mutations (up to 30%) and are excellent targets for adenovirus-based p53-mediated sensitization therapy and related forms of p53-mediated sensitization therapy. Intra-arterial delivery of the p53 vector would enable high efficiency delivery of wild-type p53 therapy-sensitizing activity into the tumor. However, systemic delivery of p53 gene for clinical benefits may not be required in many cases because hepatocellular carcinomas and head and neck cancers often produce localized morbidity as in the case of glioblastoma. Liver metastases of colorectal carcinoma and other tumors with p53 mutations could be similarly treated by intra-arterial infusion of a p53 vector followed by appropriate tumor therapy known to those skilled in the art of cancer treatment.

9. Treatment of Lung Cancer by p53-mediated Sensitization Therapy

Lung epithelium is also an excellent target for adenovirus-based p53-mediated sensitization therapy. Small cell lung carcinoma, which is initially very sensitive to chemotherapy, acquires resistance with disease progression. Introduction of wild-type p53 can be used to treat this tumor by sensitizing the tumor cells to therapy. Non-small cell lung carcinoma, also characterized by p53 mutations in some 50% of cases, is often refractory to chemotherapy. Therefore, p53-mediated sensitization therapy can be utilized in the treatment of these tumors.

### EXAMPLE 7.

Screening for Small Molecules with Therapy Sensitizing Activity

Small molecules with therapy sensitizing are identified by their ability to enhance cancer treatment efficacy relative to control solutions that do not contain the candidate small molecule. Each candidate molecule is tested for its efficacy in sensitizing cancer therapy in cell lines, in animal models, and in controlled clinical studies using methods known to those skilled in the art and approved by the Food and Drug Administration, such as, but not limited to, those promulgated in The Federal Register 47 (no. 56): 12558-12564, Mar. 23, 1982. The small molecules with therapy sensitizing or enhancing activity may be utilized in cancer therapy employing the approaches described previously for proteins with wild-type therapy-sensitizing activity. As small molecules readily diffuse into tissues following administration, this approach may be utilized to treat both localized and metastatic tumors in combination with other therapies.

Small molecules which mimic or confer wild type therapy-sensitizing activity can be screened in binding assays with the appropriate target. (See Houghten, R. A. “Peptide libraries, criteria and trends.” Trends in Genetics 9:235239, 1993). Combinatorial libraries of peptides, modified peptides or organic chemical compounds are generated by methods known to those skilled in the art (Jayarickreme et al., “Creation and functional screening of a multi-use peptide library” Proc. Natl. Acad. Sci. USA, 91:1614-1618; Houghten, R. A. “Peptide libraries, criteria and trends.” Trends in Genetics 9:235-239, 1993; Phillips et al., “Transition-state characterization; a new approach combining inhibitor analogues and variation in enzyme structure.” Biochemistry, 1992, 31(4):959-63; Eichler and Houghten, “Identification of substrate-analog trypsin inhibitors through the screening of synthetic peptide combinatorial libraries.” Biochemistry 32:11035-11041, 1993; Huston et al., “Medical applications of single-chain antibodies.” International Reviews of Immunology, 1993, 10(2-3):195-217; Van de Waterbeemd H., “Recent progress in QSAR-technology,” Drug Design and Discovery, 1993, 9(3-4):277-85).

Putative small molecules can also be analyzed in biological assays for function. In a specific example, a retroviral vector library encoding and expressing peptides could be directly screened for therapy sensitizing activity using the methods described in examples above and that of Gudkov et al., 1993, “Isolation of genetic suppressor elements, inducing resistance to topoisomerase II interactive cytotoxic drugs, from human topoisomerase II CDNA,” Proc. Natl. Acad. Sci. USA, 90:3231-3235, incorporated by reference herein.

### EXAMPLE 8.

### Toxicity-testing of Putative Therapy Sensitizing Molecules

Methods are provided for determining whether an agent active in any of the methods listed above has little or no effect on healthy cells. Such agents are then formulated in a pharmaceutically acceptable buffer or in buffers useful for standard animal tests.

By “pharmaceutically acceptable buffer” is meant any buffer which can be used in a pharmaceutical composition prepared for storage and subsequent administration, which comprise a pharmaceutically effective amount of an agent as described herein in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985). Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

A. Additional screens for Toxicity: Method 1

Agents identified as having therapy-sensitizing activity are assessed for toxicity to cultured human cells. This assessment is based on the ability of living cells to reduce 2,3,-bis[2-methoxy-4-nitro-5-sulphonylphenyl]-5-[(phenylamino)carbonyl]-2H-tetrazolium hydroxide] otherwise referred to as XTT (Paull et al., J. Heterocyl. Chem. 25:763767 (1987); Weislow et al., (1989), J. Natl. Canc. Inst. 81:577). Viable mammalian cells are capable of reductive cleavage of an N—N bond in the tetrazole ring of XTT to form XTT formazan. Dead cells or cells with impaired energy metabolism are incapable of this cleavage reaction. The extent of the cleavage is directly proportional to the number of living cells tested.

Cells from a human cell line such as HeLa cells are seeded at 10³ per well in 0.1 ml of cell culture medium (Dulbecco's modified minimal essential medium supplemented with 10% fetal calf serum) in the wells of a 96 well microtiter plate. Cells are allowed to adhere to the plate by culture at 37° C. in an atmosphere of 95% air, 5% CO₂. After overnight culture, solutions of test substances are added in duplicate to wells at concentrations that represent eight half-decade log dilutions. In parallel, the solvent used to dissolve the test substance is added in duplicate to other wells. The culture of the cells is continued for a period of time, typically 24 hours. At the end of that time, a solution of XTT and a coupler (methylphenazonium sulfate) is added to each of the test wells and the incubation is continued for an additional 4 hours before the optical density in each of the wells is determined at 450 nm in an automated plate reader. Substances that kill mammalian cells, or impair their energy metabolism, or slow their growth are detected by a reduction in the optical density at 450 nm in a well as compared to a well which received no test substance.

B. Additional screens for Toxicity: Method 2

Therapy sensitizing molecules are tested for cytotoxic effects on cultured human cell lines using incorporation of ³⁵S methionine into protein as an indicator of cell viability. HeLa cells are grown in 96 well plates in Dulbecco's minimal essential medium supplemented with 10% fetal calf serum and 50 μg/ml penicillin and streptomycin. Cells are initially seeded at 10³ cells/well, 0.1 ml/well. Cells are grown for 48 hrs without exposure to the therapy sensitizing molecule, then medium is removed and varying dilutions of the therapy-sensitizing molecule prepared in complete medium are added to each well, with control wells receiving no cytokine modulator. Cells are incubated for an additional 48-72 hrs. Medium is changed every 24 hrs and replaced with fresh medium containing the same concentration of the therapy sensitizing molecules. Medium is then removed and replaced with complete medium without antifungal. Cells are incubated for 24 hr in the absence of therapy sensitizing molecule, then viability is estimated by the incorporation of ³⁵S into protein. Medium is removed, replaced with complete medium without methionine, and incubated for 30 min. Medium is again removed, and replaced with complete medium without methionine but containing 0.1 μCi/ml ³⁵S methionine. Cells are incubated for 3 hrs. Wells are washed 3 times in PBS, then cells are permeabilized by adding 100% methanol for 10 min. Ice cold 10% trichloroacetic acid (TCA) is added to fill wells; plates are incubated on ice for 5 min. This TCA wash is repeated two more times. Wells are again washed in methanol, then air dried. 50 μl of scintillation cocktail are added to each well and dried onto the wells by centrifugation. Plates are used to expose X ray film. Densitometer scanning of the autoradiogram, including wells without antifungal, is used to determine the dosage at which 50% of cells are not viable (ID₅₀) (Culture of Animal Cells. A manual of basic technique. (1987). R. Ian Freshney. John Wiley & Sons, Inc., New York).

### EXAMPLE 9.

### Administration of Therapy Sensitizing Molecules

The invention features novel therapy sensitizing molecules discovered by the methods described above. It also includes novel pharmaceutical compositions which include therapy sensitizing molecules discovered as described above formulated in pharmaceutically acceptable formulations.

By “therapeutically effective amount” is meant an amount that relieves (to some extent) one or more symptoms of the disease or condition in the patient. Additionally, by “therapeutically effective amount” is meant an amount that returns to normal, either partially or completely, physiological or biochemical parameters associated with or causative of a mycotic disease or condition. Generally, it is an amount between about 1 nmole and 1 μmole of the molecule, dependent on its EC₅₀ and on the age, size, and disease associated with the patient.

All publications referenced are hereby incorporated by reference herein, including the nucleic acid sequences and amino acid sequences listed in each publication.

Other embodiments are within the following claims.

1. Method of increasing the effect of a cancer therapy, comprising the steps of:
delivering wild-type therapy-sensitizing gene activity to a tumor cell characterized by loss of said wild-type therapy-sensitizing gene activity, and
subjecting said tumor cell to said cancer therapy.

2. The method of claim 1, wherein a portion of a therapy-sensitizing protein with said therapy-sensitization gene activity is introduced into the tumor cell.

3. The method of claim 1, wherein a portion of a therapy-sensitizing gene or a portion of a cDNA encoding said therapy-sensitizing gene activity is introduced into the tumor cell.

4. The method of claim 1 wherein said cancer therapy is radiation therapy.

5. The method of claim 1 wherein said cancer therapy is chemotherapy.

6. The method of claim 1, wherein said cancer therapy is biological therapy.

7. The method of claim 1, wherein said cancer therapy is cryotherapy.

8. The method of claim 1, wherein said cancer therapy is hyperthermia.

9. The method of claim 1 wherein said tumor cell is selected from the group consisting of carcinoma cells, sarcoma cells, central nervous system tumor cells, melanoma tumor cells, leukemia cells, lymphoma tumor cells, hematopoietic tumor cells, ovarian carcinoma cells, osteogenic sarcoma cells, lung carcinoma cells, colorectal carcinoma cells, hepatocellular carcinoma cells, glioblastoma cells, prostate cancer cells, breast cancer cells, bladder cancer cells, kidney cancer cells, pancreatic cancer cells, gastric cancer cells, esophageal cancer cells, anal cancer cells, biliary cancer cells, urogenital cancer cells, and head and neck cancer cells.

10. The method of claim 3 wherein said portion of a therapy-sensitizing gene or said portion of a cDNA is in a vector.

11. The method of claim 10, wherein said vector is selected from the group consisting of adenovirus vector, retroviral vector, adeno-associated virus vector, herpes virus vector, vaccinia virus vector and papilloma virus vector.

12. The method of claim 3, wherein said portion of a therapy-sensitizing gene or said portion of a cDNA is coupled to a virus capsid or particle.

13. The method of claim 12, wherein said portion of a therapy-sensitizing gene or said portion of a cDNA is coupled to said capsid or particle through a polylysine bridge.

14. The method of claim 3, wherein said portion of a therapy-sensitizing gene or said portion of a cDNA is encapsulated in a liposome.

15. The method of claim 3, wherein said portion of a therapy-sensitizing gene or said portion of a cDNA is conjugated to a ligand.

16. The method of claim 15, wherein said ligand is an asialoglycoprotein.

17. The method of claim 3, wherein said portion of a therapy-sensitizing gene or said portion of a cDNA is introduced to said tumor cell by direct injection or aerosolized preparation.

18. The method of claim 3, wherein said portion of a therapy-sensitizing gene or said portion of a cDNA is introduced to said tumor cell by intra-arterial infusion.

19. The method of claim 3, wherein said portion of a therapy-sensitizing gene or said portion of a cDNA is introduced to said tumor cell by intracavitary infusion.

20. The method of claim 3, wherein said portion of a therapy-sensitizing gene or said portion of a cDNA is introduced to said tumor cell by intravenous infusion.

21. The method of claim 1, wherein said therapy-sensitizing gene activity is fas therapy-sensitizing activity.

22. The method of claim 1, wherein said therapy-sensitizing gene activity is p53 therapy-sensitizing activity.
